(19) United States
(12) Patent Application Publication
(10) Pub. No.: US 2011/0190651 A1
(43) Pub. Date: Aug. 4, 2011

(54) APPARATUS AND METHOD FOR APNEA DETECTION

(75) Inventors: Yasuji Ota, Kawasaki (JP); Kaori Endo, Kawasaki (JP); Takeshi Otani, Kawasaki (JP); Taro Togawa, Kawasaki (JP)

(73) Assignee: FUJITSU LIMITED, Kawasaki-shi (JP)

(21) Appl. No.: 13/085,737

(22) Filed: Apr. 13, 2011

Related U.S. Application Data
(63) Continuation of application No. PCT/JP2008/068760, filed on Oct. 16, 2008.

(51) Int. Cl. A61B 5/11

(52) U.S. Cl. 600/534

(57) ABSTRACT

In an apnea detecting apparatus, a respiratory pace analyzer determines a respiratory pace of a user based on an ambient noise (including a breathing sound, a snore, and the like of the user) generated in a surrounding area and on information of a posture of the user in determining whether or not the user suffers from the apnea syndrome. Then, an apnea time extracting unit in the apnea detecting apparatus determines whether or not a silent time is included in a time period during which the user is estimated to be breathing based on the respiratory pace and the silent time in the ambient noise.

### CROSS-REFERENCE TO RELATED APPLICATION

This application is a continuation of PCT international application Ser. No. PCT/JP2008/068760 filed on Oct. 16, 2008 which designates the United States, incorporated herein by reference.

### FIELD

The embodiment discussed herein is directed to an apparatus and a method for detecting an apnea state of a user.

### BACKGROUND

Recently, a polysomnography (hereinafter referred to as “PSG”) has been known as a technique of detecting an apnea state during sleep. The PSG is a technique of performing an examination to detect a state of an apnea during sleep via a measurement of an electroencephalogram, an electro-oculogram, a respiratory movement of a diaphragm and an abdominal muscle, and the like. However, since the PSG requires an examination while performing the measurement of an electroencephalogram, an electro-oculogram, a respiratory movement of a diaphragm and an abdominal muscle, and the like, there has been a problem of a restriction in location and an expense in time and cost.

In response, there is an apnea detector provided with a respiratory movement sensor of a pressure sensitive type, an audio sensor, a recorder, and a data processor known as a portable apnea detector. This apnea detector records data collected by the respiratory movement sensor and the audio sensor in the recorder, calculates an index value, and uses the calculated index value to provide a diagnosis of an apnea. Detailed information of this technique can be obtained in Japanese Laid-open Patent Publication No. H10-295695, for example.

In addition, there is another technique of measuring a respiratory sound during sleep and visualizing a result of the measured respiratory sound known as a technique of checking a condition of a respiration during sleep. Detailed information of this technique can be obtained in Japanese Laid-open Patent Publication No. 2004-33254. In this technique, a specialist will visually check a condition of the visualized respiratory sound to examine a sleep apnea.

However, there has been a problem of not being able to detect an apnea state during sleep easily and at low cost in the above-described conventional techniques.

For example in the technique of recording data collected by the respiratory movement sensor and the audio sensor in the recorder and calculating an index value, there is a problem of having a large-scale structure of devices and imposing a large load on a user though the problem in location, time, and the like is eased slightly compared to the conventional PSG. In addition, there is another problem of requiring a cost since an actual examination by a specialist is required for a detection (diagnosis) of an apnea state during sleep.

Besides, since a respiratory sound is only visualized and no analysis on a possibility of a sleep apnea syndrome is performed in the technique of measuring a respiratory sound and visualizing a result described above, a specialist who examines a condition of the visualized respiratory sound for the detection (diagnosis) of an apnea state during sleep is required, which causes an expense for the examination by the specialist and the like.

### SUMMARY

According to an aspect of an embodiment of the invention, an apnea detecting apparatus includes an ambient noise recorder that records an ambient noise generated from a surrounding area; a respiratory pace determining unit that determines, after obtaining information of a posture of a user from a posture determining unit that determines the posture of the user, a respiratory pace of the user based on the information of the posture and on a noise time and a silent time in the ambient noise when the user lies on a back; and an apnea detector that detects an apnea state of the user by determining whether or not the silent time is included in a time period during which the user is estimated to be breathing based on the silent time in the ambient noise and the respiratory pace.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is an explanatory view of a summary and characteristics of an apnea detecting apparatus according to an embodiment;

FIG. 2 is a functional block diagram of a configuration of the apnea detecting apparatus according to the embodiment;

FIG. 3 illustrates a spectrum property of a snore time;

FIG. 4 is an explanatory view of one example of a process performed by a respiratory pace analyzer;

FIG. 5 illustrates one display example of a result of a sleep apnea detection;

FIG. 6 illustrates another display example of a result of a sleep apnea detection;

FIG. 7 illustrates one example of a system configuration;

FIG. 8 is a flowchart of a procedure of a process performed by the apnea detecting apparatus according to the embodiment;

FIG. 9 is a flowchart of a procedure of an apnea analyzing process; and

FIG. 10 illustrates a hardware configuration of a computer constituting the apnea detecting apparatus according to the embodiment.

### DESCRIPTION OF EMBODIMENT

A preferred embodiment of an apnea detecting program, an apnea detecting apparatus, and an apnea detecting method will be explained in detail below with reference to the accompanying drawings.

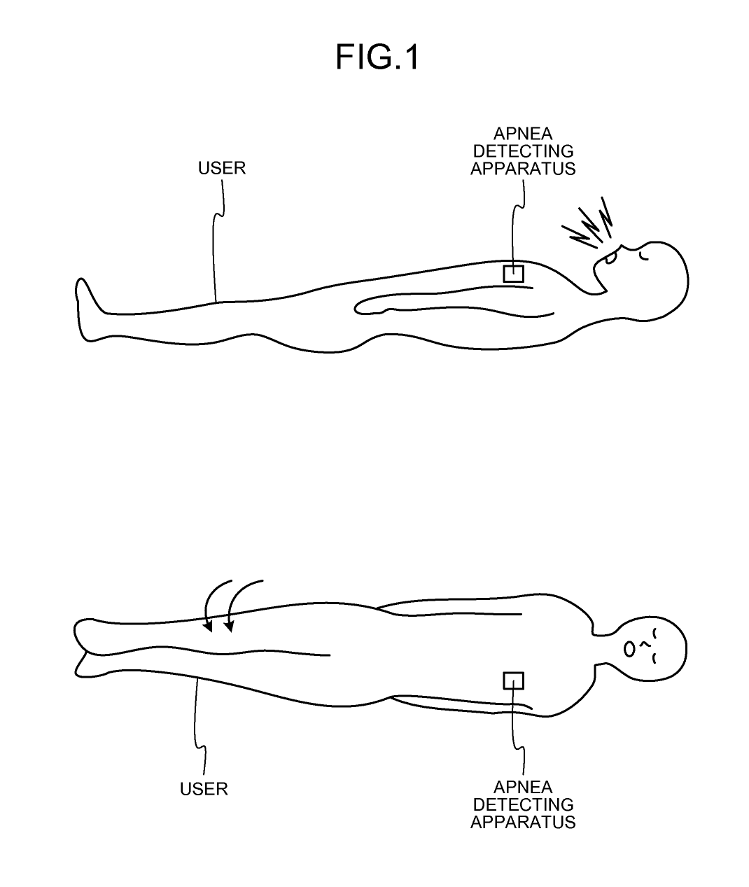

First, a summary and characteristics of an apnea detecting apparatus according to an embodiment will be explained. FIG. 1 is an explanatory view of a summary and characteristics of an apnea detecting apparatus according to an embodiment. As an example in this embodiment, an apnea detecting apparatus is configured to be arranged in a chest pocket and the like of a user and detect an apnea state of the user.

It is medically known as characteristics of a human body that a snore and an apnea tend to occur easily when a user lies on his/her back as illustrated at an upper part in FIG. 1 and a snore stops when the user turns over as illustrated at a lower part in FIG. 1 (or the user lies on his/her side). The apnea detecting apparatus according to the embodiment detects an apnea state of a user based on a focus on these points.

In determining whether or not a user suffers from an apnea syndrome, the apnea detecting apparatus according to the embodiment determines a respiratory pace of the user based on an ambient noise (including a breathing sound, a snore, and the like of the user) generated from a surrounding area and on information of a posture of the user.

Specifically, the apnea detecting apparatus determines a respiratory pace of the user based on a period interval and a time interval of a noise time and a silent time in the ambient noise when a property of a snore is included in the ambient noise since the user is more likely to snore in lying on his/her back.

In contrast, the apnea detecting apparatus does not use the ambient noise when the user lies on his/her side since the user is less likely to snore in lying on his/her side and there is a high possibility that the ambient noise may include a noise (a friction noise) in turning over.

The apnea detecting apparatus detects an apnea state of the user by determining whether or not a silent time is included in a time period in which the user is estimated to be breathing based on the respiratory pace and the silent time in the ambient noise.

In this manner, since determining a respiratory pace of the user based on the posture information of the user, the apnea detecting apparatus according to the embodiment can precisely determine a respiratory pace of a user by eliminating an influence of a noise and the like and detect an apnea state during sleep easily and at low cost.

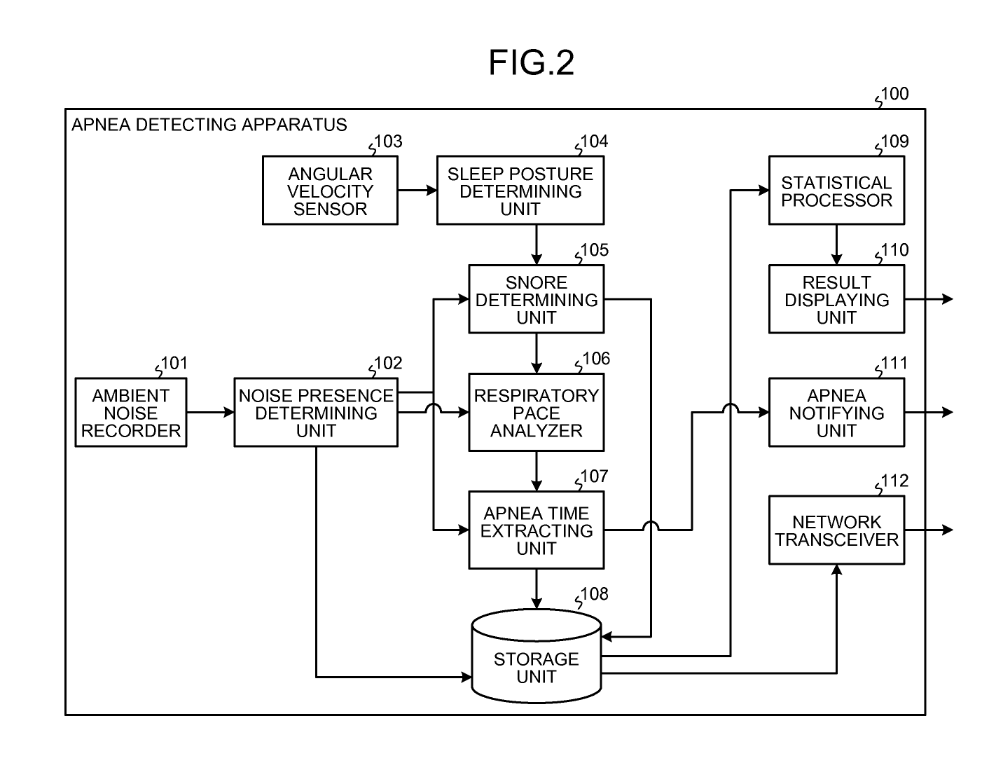

Next, a configuration of the apnea detecting apparatus according to the embodiment will be explained. FIG. 2 is a functional block diagram of a configuration of the apnea detecting apparatus according to the embodiment. As illustrated in FIG. 2, an apnea detecting apparatus 100 is provided with an ambient noise recorder 101, a noise presence determining unit 102, an angular velocity sensor 103, a sleep posture determining unit 104, a snore determining unit 105, a respiratory pace analyzer 106, an apnea time extracting unit 107, a storage unit 108, a statistical processor 109, a result displaying unit 110, an apnea notifying unit 111, and a network transceiver 112.

Among these units, the ambient noise recorder 101 records an ambient noise generated in a surrounding area of the apnea detecting apparatus 100 and includes a microphone, for example. The ambient noise recorder 101 outputs the ambient noise recorded by using a microphone and the like to the noise presence determining unit 102.

The noise presence determining unit 102 determines a noise time and a silent time included in the ambient noise. The noise presence determining unit 102, for example, divides the ambient noise into time frames, analyzes a noise property amount for each time frame, determines whether or not a noise is present, and determines the noise time and the silent time. When the noise time is determined in the ambient noise, the noise presence determining unit 102 outputs data of the noise time and the ambient noise to the snore determining unit 105.

Besides, the noise presence determining unit 102 determines a timing from which an ambient noise included in each time frame becomes silent and a silent time and outputs data of the determined timing, the silent time, and the ambient noise to the apnea time extracting unit 107. The noise presence determining unit 102 may determine a state in which no noise is present to be silent or may determine a minor noise the level of which is equal to or less than a predetermined threshold value to be silent. In addition, the noise presence determining unit 102 outputs the data of the noise time, the silent time, and the ambient noise to the respiratory pace analyzer 106.

The angular velocity sensor 103 measures an angular velocity of the apnea detecting apparatus 100. The angular velocity sensor 103 outputs data of the measured angular velocity to the sleep posture determining unit 104.

The sleep posture determining unit 104 determines a posture of the user based on the angular velocity output from the angular velocity sensor 103. The sleep posture determining unit 104, for example, determines that the user lies on his/her back when the angular velocity is less than a threshold value, determines that the user lies on his/her side when the angular velocity is equal to or more than the threshold value, and outputs the determination result to the snore determining unit 105. Here, the method of determining the posture of the user is not limited to that described above and any conventional techniques may be adopted to determine the posture of the user.

The snore determining unit 105 determines whether or not a property of a snore (breathing sound) during sleep is included in the data of the ambient noise in the noise time. To explain a process of the snore determining unit 105 specifically, the snore determining unit 105 first obtains a result of the determination by the sleep posture determining unit 104 and determines whether the user lies on his/her back or on his/her side.

when the user lies on his/her back, the snore determining unit 105 compares a frequency property of the ambient noise corresponding to the noise time to a frequency property of a snore and determines that the snore property is included in the noise time when an agreement rate of each frequency property is equal to or more than a threshold value. Here, the frequency property of a snore is configured to be retained in the snore determining unit 105.

When the user lies on his/her side, the snore determining unit 105 determines the snore property is not included in the ambient noise corresponding to the noise time. Alternatively, the snore determining unit 105 may eliminate an effect of a noise by decreasing a sensitivity to an ambient noise, compare a frequency property of the ambient noise from which an effect of a noise is eliminated to the frequency property of a snore, and determine that the snore property is included in the noise time when an accordance rate of each frequency property is equal to or more than the threshold value.

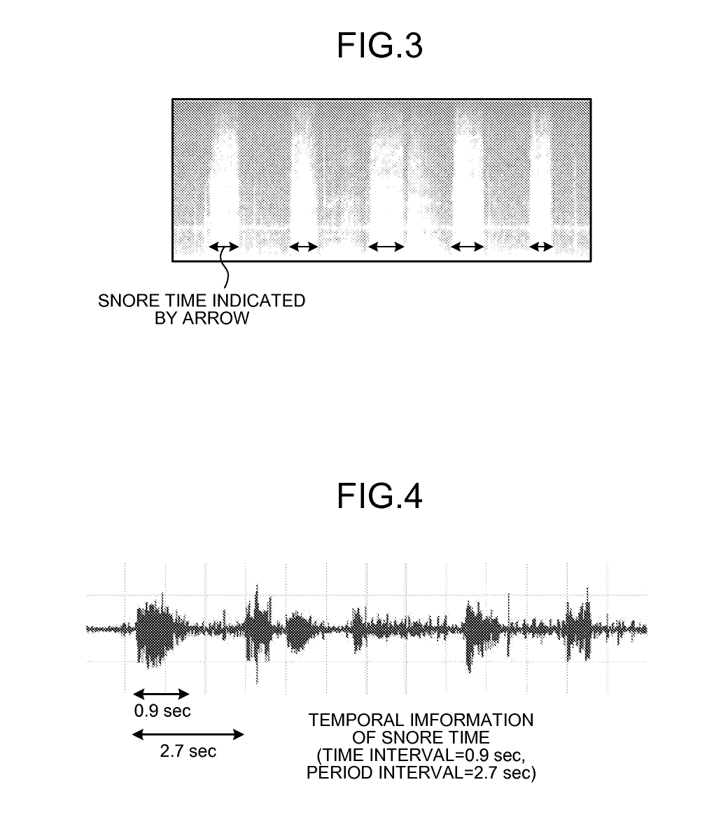

The snore determining unit 105 outputs, when determining that the snore property is included in the noise time, an ambient noise of the noise time including the snore property to the respiratory pace analyzer 106. In the explanation below, the noise time including the snore property will be described as a “snore time”. FIG. 3 illustrates a spectrum property of a snore time. A time period indicated by an arrow in FIG. 3 is the snore time.

The respiratory pace analyzer 106 uses the noise time (snore time), the silent time, and the ambient noise and analyzes a respiratory pace which indicates a period of an “estimated snore time” during which breathing (snoring) is estimated to be performed. The respiratory pace analyzer 106 outputs an analysis result to the apnea time extracting unit 107. FIG. 4 is an explanatory view of one example of a process performed by the respiratory pace analyzer 106.

In an example illustrated in FIG. 4, the respiratory pace analyzer 106 calculates “0.9 sec.” for a time interval of the snore time and “2.7 sec.” for a period interval of the snore time. Here, to ensure a statistical accuracy, the respiratory pace analyzer 106 may perform averaging by a predetermined time in calculating the time interval and the period interval.

For example, the respiratory pace analyzer 106 can calculate a respiratory pace (Freqₐᵥₑ) by

Freqₐᵥₑ=60U/Pₐᵥₑ (1)

The denotation “Pₐᵥₑ” (sec.) in equation (1) is an average value of the period interval and the denotation “U” is a value which indicates how many minutes the unit of the respiratory pace is. When a numeral “1” is assigned to the denotation “U” for example, a respiratory pace by the minute is calculated by equation (1).

The apnea time extracting unit 107 detects an apnea state of the user based on the timing from which the silent time starts, the time interval, and the analysis result of the respiratory pace of the user. A process of the apnea time extracting unit 107 will be specifically explained below.

The apnea time extracting unit 107 first calculates a time period during which a snore is estimated (hereinafter referred to as an “estimated snore time”) based on the respiratory pace and the time interval of a snore and determines whether or not a silent time determined to be silent is present in the estimated snore time. The apnea time extracting unit 107 then determines a snore disruption when a silent time is present in the estimated snore time.

As illustrated in FIG. 4, for example, while the user snores for about 0.9 sec. in each about 2.7 sec. at a normal time, the apnea time extracting unit 107 determines a snore disruption when a silent time is present in a manner of overlapping the snore timing of each about 0.9 sec. (when the silent time is present at a timing from which snoring is expected to start).

When determining the snore disruption, the apnea time extracting unit 107 then determines whether the snore disruption is an incident or caused by an apnea syndrome. For example, the apnea time extracting unit 107 determines that the user is in an apnea state when the snore disruption is detected more than predetermined number of times within a time period which an administrative person defines.

The apnea time extracting unit 107, when determining that the user is in an apnea state, outputs information to the effect that the user is in an apnea state to the apnea notifying unit 111. In addition, the apnea time extracting unit 107 a result of the determination to the storage unit 108.

The storage unit 108 stores data such as the result of the determination by the apnea time extracting unit 107, the result of the determination by the snore determining unit 105, and the ambient noise output from the noise presence determining unit 102.

The statistical processor 109 reads out the result of the determination of the apnea time from the storage unit 108 and performs a statistical process. Specifically, the statistical processor 109 reads out the result of the apnea time extraction from the storage unit 108, performs a statistical process, calculates a possibility of the apnea syndrome, and notifies the calculation result to the result displaying unit 110. For example, the statistical processor 109 may calculate a ratio of a time period of the apnea time to an entire time period, a total time period of the apnea time, and a presence of the apnea time as information indicating the possibility of the apnea syndrome.

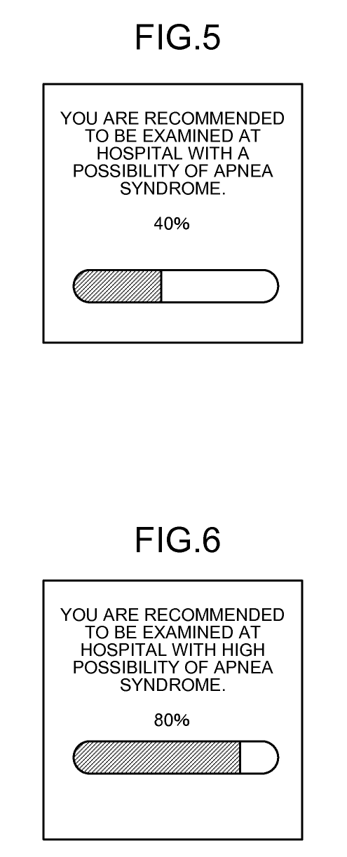

The result displaying unit 110 displays the possibility of the apnea syndrome to the user and includes a display and a speaker. For example, the result displaying unit 110 may display the possibility of the apnea syndrome in figures as illustrated in FIGS. 5 and 6. FIGS. 5 and 6 illustrate display examples of the result of the sleep apnea detection.

The result displaying unit 110 may display information of encouraging seeking a diagnosis by a specialist depending on the statistical result. To put it another way, the apnea detecting apparatus 100 displays and informs to the user of the possibility of the apnea syndrome, so that the user can be aware of the result of the diagnosis of the apnea syndrome easily.

The apnea notifying unit 111 notifies the user of the apnea state when the apnea time extracting unit 107 determines that the user is in the apnea state and includes an alarm and a vibrator. Specifically, the apnea notifying unit 111 activates an alarm and a vibrator when the user is determined to be in the apnea state.

In other words, the apnea is controlled by notifying the sleeping user of the apnea state. Here, whether or not to notify the apnea state can be set by the user and an alarm and a vibrator may be set not to be activated.

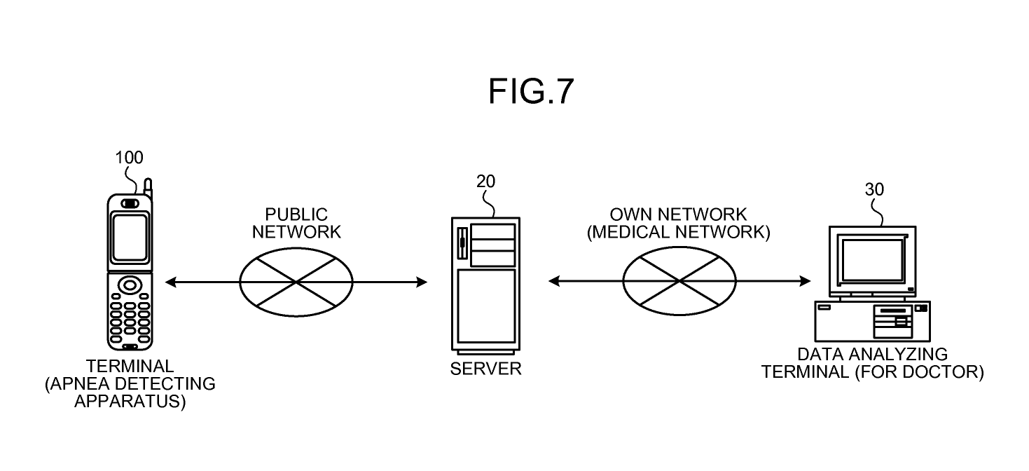

The network transceiver 112 controls a communication concerning various information which is transferred with a server connected to the apnea detecting apparatus 100 and an analyzing terminal. FIG. 7 illustrates one example of a system configuration. As illustrated in FIG. 7, the apnea detecting apparatus 100 is connected to a server 20 via a public network and the server 20 is connected to an analyzing terminal (a terminal used by a doctor for a diagnosis, for example) 30 via its own network (a medical network, for example).

For example, the network transceiver 112 transmits, to the data analyzing terminal 30 via the server 20, the result of the extraction of the apnea time stored in the storage unit 108 as statistical data. Briefly, when the result of the detection of the apnea time stored in the storage unit 108 as statistical data to the data analyzing terminal 30, the apnea detecting apparatus 100 can utilize the statistical data as supplementary information obtained from the data analyzing terminal 30 in making a diagnosis by a doctor to assist a diagnosis of a sleep apnea syndrome.

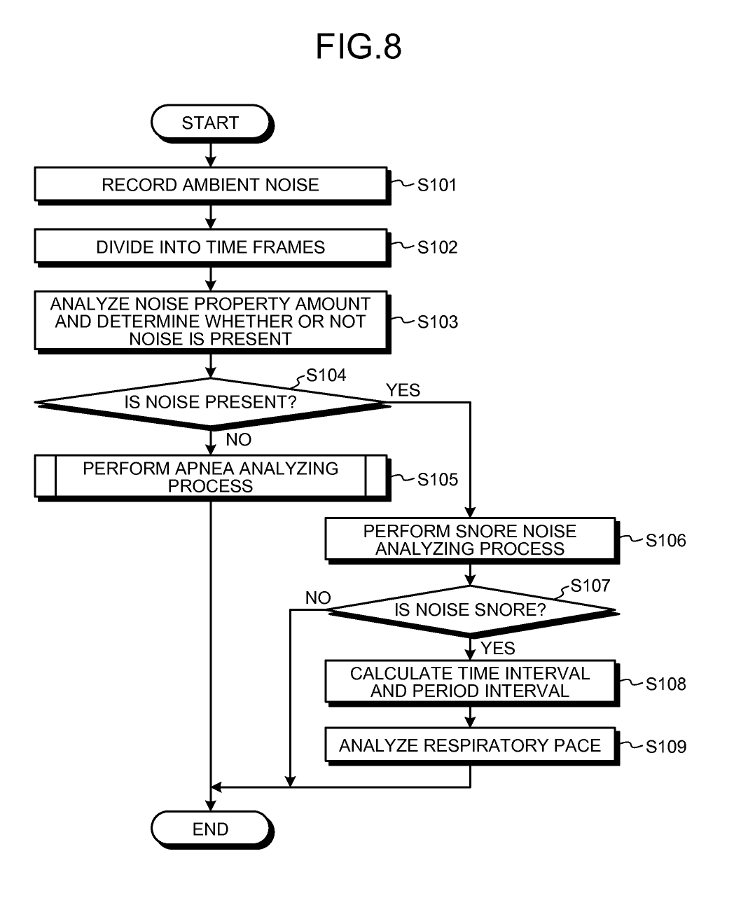

Next, a procedure of a process performed by the apnea detecting apparatus 100 according to the embodiment will be explained. FIG. 8 is a flowchart of a procedure of a process performed by the apnea detecting apparatus 100 according to the embodiment. As illustrated in FIG. 8, the ambient noise recorder 101 records an ambient noise (step S101), and the noise presence determining unit 102 divides data of the ambient noise into predetermined time frames (step S102) and determines whether or not a noise is present via an analysis on the noise property amount (step S103) in the apnea detecting apparatus 100.

When it is determined that no noise is present (“No” at step S104), the apnea detecting apparatus 100 then executes an apnea analyzing process (step S105). On the other hand when it is determined that a noise is present (“Yes” at step S104), the apnea detecting apparatus 100 executes a snore noise analyzing process (step S106).

When it is determined that the user lies on his/her back and the snore property is included in the ambient noise (“Yes” at step S107), the apnea detecting apparatus 100 calculates time information including a silent time, and the time interval and the period interval of the snore time (step S108) and analyzes the respiratory pace of the user (step S109).

On the other hand when it is determined that the user lies on his/her side and the snore property is not included in the ambient noise (“No” at step S107), the apnea detecting apparatus 100 ends the process.

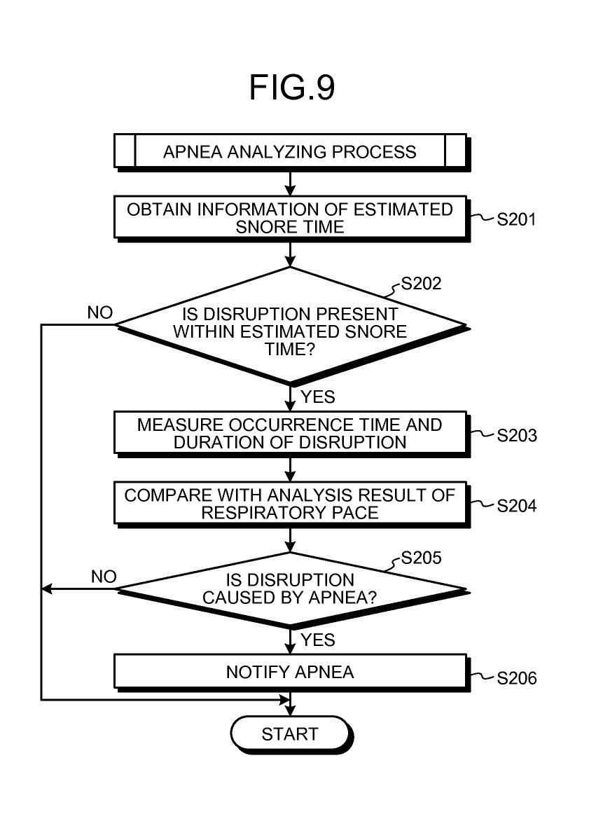

Next, a procedure of the apnea analyzing process explained at step S105 in FIG. 8 will be explained. FIG. 9 is a flowchart of a procedure of the apnea analyzing process. As illustrated in FIG. 9, the apnea detecting apparatus 100 obtains information of the estimated snore time based on the analyzed respiratory pace and the time interval of the snore time (step S201) and determines whether or not the silent time determined to be silent is within the estimated snore time (step S202).

When it is determined that the silent time is not included in the estimated snore time, i.e., the silent time is not a disruption of the estimated snore time (“No” at step S202), the apnea detecting apparatus 100 ends the apnea analyzing process. On the other hand, when the silent time is a disruption within the estimated snore time (“Yes” at step S202), the apnea detecting apparatus 100 measures an occurrence time and a duration of the disruption (step S204) and determines whether the snore disruption of is due to an apnea or simply a disappearance of the snore (step S205).

When it is determined that the snore disruption is caused by an apnea (“Yes” at step S205), the apnea detecting apparatus 100 notifies the user of the apnea (step S206), and ends the process. On the other hand when it is determined that the snore disruption is not an apnea (“No” at step S205), the apnea detecting apparatus 100 treats the snore disruption not as an apnea but just as a disappearance of the snore and directly ends the process.

As described so far, the apnea detecting apparatus 100 according to the embodiment determines a respiratory pace of a user based on an ambient noise (including a breathing sound, a snore, and the like of the user) generated in a surrounding area and on information of a posture of the user in determining whether or not the user suffers from the apnea syndrome. Since the apnea detecting apparatus 100 determines, to detect an apnea state of the user, whether or not the silent time is included in a time period during which the user is estimated to be breathing based on the respiratory pace and the silent time in the ambient noise, an apnea state during sleep can be detected easily and at low cost.

By the way, all or a part of the processes explained in the embodiment as being performed automatically can be performed manually, or all or a part of the processes explained as being performed manually can be performed automatically in a known method. Furthermore, a process procedure, a control procedure, a specific name, and information including various data and parameters described in the description and the drawings can be arbitrarily modified unless otherwise specified.

Besides, each of the constituents of the apnea detecting apparatus 100 illustrated in FIG. 2 are only exemplary and explanatory on a functional and conceptual basis, and are not necessarily required to be configured physically as illustrated. Here, various process procedures explained in the embodiment may be realized by making a computer such as a personal computer and a workstation execute a program prepared in advance.

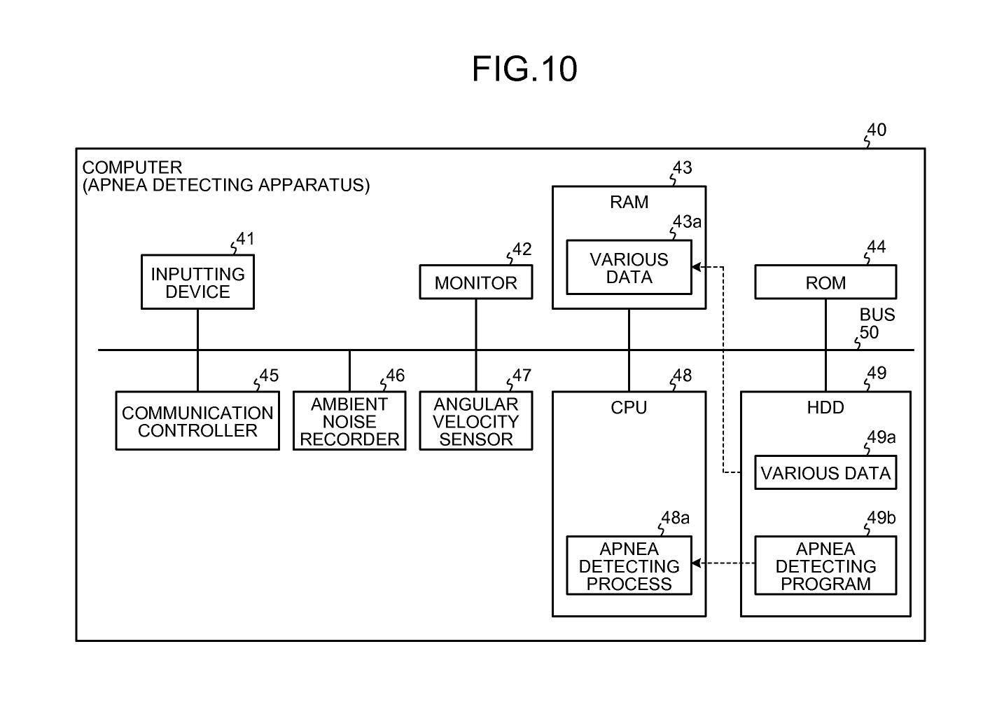

FIG. 10 illustrates a hardware configuration of a computer constituting the apnea detecting apparatus 100 according to the embodiment. As illustrated in FIG. 10, a computer (an apnea detecting apparatus) 40 is connected to an inputting device 41, a monitor 42, a random access memory (RAM) 43, a read-only memory (ROM) 44, a communication controller 45 that communicates with other devices via a network, an ambient noise recorder 46 that records an ambient noise, an angular velocity sensor 47, a central processing unit (CPU) 48, a hard disk drive (HDD) 49 via a bus 50.

In the HDD 49, an apnea detecting program 49b that exercises functions similar to the apnea detecting apparatus 100 described above is stored. When the CPU 48 reads out and executes the apnea detecting program 49b, an apnea detecting process 48a is activated.

Here, the apnea detecting process 48a is equivalent to the noise presence determining unit 102, the sleep posture determining unit 104, the snore determining unit 105, the respiratory pace analyzer 106, the apnea time extracting unit 107, the statistical processor 109, the result displaying unit 110, the apnea notifying unit 111, and the network transceiver 112 illustrated in FIG. 2. The HDD 49 stores various data 49a equivalent to the information stored in the storage unit 108 of the apnea detecting apparatus 100. The CPU 48 reads out the various data 49a stored in the HDD 49, stores the read data in the RAM 43, and detects an apnea state of the user by using the various data 43a stored in the RAM 43.

Here, the apnea detecting program 49b illustrated in FIG. 10 is not necessarily required to be stored in the HDD 49 from the beginning. For example, the apnea detecting program 49b may be stored in “portable physical media” to be inserted into a computer such as a flexible disk (FD), a CD-ROM, a DVD, an optical magnetic disk, and an IC card; “fixed physical media” such as an HDD provided in an inside or an outside of a computer; and “other computers (or a server)” to be connected to a computer via a public network, the Internet, a LAN, a WAN, and the like, and may be read out therefrom and executed by the computer.

In the apnea detecting program according to the embodiment, a respiratory pace is precisely determined based on a posture of a user, and a noise time and a silent time in an ambient noise, and an apnea state during sleep can be detected without requiring a large scale device and an examination by a doctor, so that an apnea state during sleep can be precisely detected easily and at low cost.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiments of the present inventions have been described in detail, it should be understood that the various changes, substitutions, and alterations could be made hereto without departing from the spirit and scope of the invention.

What is claimed is:

1. A computer-readable, non-transitory medium storing an apnea detecting program causing a computer to execute a process comprising:
recording an ambient noise generated from a surrounding area by an ambient noise recorder;
determining, after obtaining information of a posture of a user by a posture determining unit that determines the posture of the user, a respiratory pace of the user based on the information of the posture and on a noise time and a silent time in the ambient noise when the user lies on a back; and
detecting an apnea state of the user by determining whether or not the silent time is included in a time period during which the user is estimated to be breathing based on the silent time in the ambient noise and the respiratory pace.

2. The computer-readable, non-transitory medium according to claim 1, wherein the determining includes determining the respiratory pace based on the noise time and the silent time in the ambient noise when the user lies on the back and when a property of a snore of the user is included in the noise time in the ambient noise.

3. The computer-readable, non-transitory medium according to claim 1, wherein the determining includes determining whether or not a property of a snore of the user is included in the noise time in the ambient noise after decreasing a sensitivity to the ambient noise when the user lies on a side, and determining the respiratory pace based on the noise time and the silent time in the ambient noise when the property of the snore is included in the noise time.

4. An apnea detecting apparatus, comprising:
an ambient noise recorder that records an ambient noise generated from a surrounding area;
a respiratory pace determining unit that determines, after obtaining information of a posture of a user from a posture determining unit that determines the posture of the user, a respiratory pace of the user based on the information of the posture and on a noise time and a silent time in the ambient noise when the user lies on a back; and
an apnea detector that detects an apnea state of the user by determining whether or not the silent time is included in a time period during which the user is estimated to be breathing based on the silent time in the ambient noise and the respiratory pace.

5. The apnea detecting apparatus according to claim 4, wherein the respiratory pace determining unit determines the respiratory pace based on the noise time and the silent time in the ambient noise when the user lies on the back and when a property of a snore of the user is included in the noise time in the ambient noise.

6. The apnea detecting apparatus according to claim 4, wherein the respiratory pace determining unit determines whether or not a property of a snore of the user is included in the noise time in the ambient noise after decreasing a sensitivity to the ambient noise when the user lies on a side and determines the respiratory pace based on the noise time and the silent time in the ambient noise when the property of the snore is included in the noise time.

7. An apnea detecting method performed by an apnea detecting apparatus, the apnea detecting method, comprising:
recording an ambient noise generated from a surrounding area by an ambient noise recorder;
determining, after obtaining information of a posture of a user by a posture determining unit that determines the posture of the user, a respiratory pace of the user based on the information of the posture and on a noise time and a silent time in the ambient noise when the user lies on a back; and
detecting an apnea state of the user by determining whether or not the silent time is included in a time period during which the user is estimated to be breathing based on the silent time in the ambient noise and the respiratory pace.

8. The apnea detecting method according to claim 7, wherein the determining includes determining the respiratory pace based on the noise time and the silent time in the ambient noise when the user lies on the back and when a property of a snore of the user is included in the noise time in the ambient noise.

9. The apnea detecting method according to claim 7, wherein the determining includes determining whether or not a property of a snore of the user is included in the noise time in the ambient noise after decreasing a sensitivity to the ambient noise when the user lies on a side, and determining the respiratory pace based on the noise time and the silent time in the ambient noise when the property of the snore is included in the noise time.
